(19) United States
(12) Patent Application Publication
(10) Pub. No.: US 2010/0075424 A1
(43) Pub. Date: Mar. 25, 2010

(54) METHODS AND COMPOSITIONS FOR GENETICALLY ENGINEERING CLOSTRIDIA SPECIES

(75) Inventors: Bryan P. Tracy, Newark, DE (US); Eleftherios T. Papoutsakis, Newark, DE (US)

Correspondence Address: Casimir Jones, S.C., 2275 DEMING WAY, SUITE 310, MIDDLETON, WI, 53562, US

(73) Assignee: NORTHWESTERN UNIVERSITY, Evanston, IL (US)

(21) Appl. No.: 12/437,985

(22) Filed: May 8, 2009

Related U.S. Application Data
(60) Provisional application No. 61/051,515, filed on May 8, 2008.

(51) Int. Cl. C12N 15/74; C12N 15/01; C12N 1/21

(52) U.S. Cl. 435/477; 435/476; 435/471; 435/441; 435/252.3

(57) ABSTRACT

The present invention relates to methods and compositions for engineering Clostridia species. In particular, embodiments of the present invention relate to the expression of recombinant resolvase proteins in Clostridia species.

### CROSS REFERENCE TO RELATED APPLICATIONS

This application claims priority to application Ser. No. 61/051,515, filed May 8, 2008, which is herein incorporated by reference in its entirety.

### GOVERNMENT SUPPORT

This invention was made with government support under Grant BES-0418157 awarded by the National Science Foundation. The government has certain rights in the invention.

### FIELD OF INVENTION

The present invention relates to methods and compositions for engineering bacterial cells, particularly a cell of the class Clostridia. In particular, embodiments of the present invention relate to the expression of recombinant resolvase proteins in Clostridia.

### BACKGROUND OF THE INVENTION

The engineering of microbes for specialty chemical conversion, biofuel generation, bioremediation and pharmaceutical production remains an immediate scientific and industrial goal. Specifically for the class Clostridia among prokaryotes, the pursuit of industrial scale biofuel generation and Clostridia-based cancer therapies is motivating a tremendous amount of strain development. Clostridia are naturally some of the most prolific microorganisms for fermenting cellulosic material into valuable biofuel alcohols such as butanol and ethanol. Additionally, due to their anaerobic and spore forming characteristics, Clostridia are being engineered to target the necrotic and anaerobic cores of malignant tumors to kill tumors from the inside out.

The study of Clostridia (including both industrially useful and pathogenic strains), as well as generation of new recombinant and knock-out Clostridia strains having important industrial and therapeutic applications, would benefit from a genetic system that makes chromosomal integration easy and predictable. However, the tools for genetically manipulating Clostridia remain limiting and insufficient for harnessing the awesome potential of this important class of bacteria. Advances have occurred slowly over the past twenty years, but need to be dramatically accelerated, especially given the recent interest in biofuels. Two of the more notable limitations of current methods are engineering gene specific mutants for gene inactivation and generating genetically diverse mutant populations for genome scale library screenings.

What is needed are improved strategies for engineering Clostridia and other bacterial species that are difficult to engineer.

### SUMMARY OF THE INVENTION

The present invention relates to methods and compositions for engineering Clostridia species. In particular, embodiments of the present invention relate to the expression of recombinant resolvase proteins in Clostridia species.

Embodiments of the present invention provide compositions, kits, and methods for incorporating exogenous resolvase activity into bacterial cells lacking native resolvase activity (e.g., Clostridia) for the purpose of promoting recombination in the cell. For example, in some embodiments, the present invention provides a method for incorporating genetic material into a bacterial genome, wherein the bacterial genome lacks a functional resolvase gene, comprising: contacting a bacterial cell comprising a bacterial genome with at least one plasmid comprising a gene encoding a resolvase protein and a nucleic acid of interest under conditions such that the nucleic acid of interest integrates into the bacterial genome. In some embodiments, the gene encoding a resolvase protein and the nucleic acid of interest are on the same plasmid or on two distinct plasmids. In some embodiments, the nucleic acid of interest integrates into the bacterial genome via homologous recombination (e.g., site specific recombination). In some embodiments, the integration of the nucleic acid of interest into the bacterial genome results in disruption of function of one or more genes in the bacterial genome. In some embodiments, the resolvase polypeptide is encoded by the recU gene from Bacillus subtilis (e.g., SEQ ID NO:25). In other embodiments, the nucleic acid or interest encodes a protein having an amino acid sequence of any of SEQ ID NOs: 26-33. In some embodiments, the resolvase gene is under the control of a Clostridia promoter (e.g., Clostridium thiolase (thL) or phosphotransbutyrylase (ptB) promoters). In some embodiments, the nucleic acid of interest also encodes a selectable marker (e.g., an antibiotic resistance gene).

Further embodiments of the present invention provide a method, comprising: contacting a bacterial cell comprising a bacterial genome lacking a native resolvase gene with a nucleic acid encoding an exogenous resolvase gene under conditions such that the exogenous resolvase gene is stabily incorporated into the bacterial cell (e.g., as a plasmid or via incorporation into the genome). In some embodiments, the method further comprises the step of contacting the bacterial genome with a sub-lethal concentration of a reagent that induces mutation, and optionally, the additional step of selecting for bacterial cells that grow in the presence of the reagent.

Additional embodiments of the present invention provide a bacterial cell comprising an exogenous nucleic acid encoding a resolvase protein (e.g., as a plasmid or incorporated into the genome). In some embodiments, the bacterial cell lacks a native resolvase gene. In some embodiments, the resolvase gene has the nucleic acid sequence of SEQ ID NO:25 or encodes a protein having an amino acid sequence of any of SEQ ID NOs: 26-33.

### DESCRIPTION OF THE FIGURES

FIG. 1: Mechanism for homologous recombination in C. acetobutylicum. Illustration of a commonly accepted mechanism for homologous recombination in gram-positive bacteria. The gene numbers from the annotated C. acetobutylicum ATCC824 genome for the essential proteins involved are given in parentheses.

FIG. 2: Campbell-like double crossover recombination for targeted chromosomal integration. Campbell-like double crossover homologous recombination involves two homologous recombination events. The first recombines one region of homology with the chromosome, thus integrating the entire plasmid into the chromosome. The second recombination event occurs between the other region of homology and the chromosome, resulting in the excision of the plasmid components outside of the regions of homology. In the schematic this results in the integration of the MLRs cassette and excision of Ori, repL, CM resistance gene, and rec.

FIG. 3: Specific experimental approach for utilizing recU expression towards enhancing homologous recombination efficiency. The 500 bp region of the spo0A gene (CAC2071) that is targeted for disruption via chromosomal integration is shown. ORI, origin of replication for gram negative bacteria; repL, origin of replication for gram positive bacteria; recU, recU gene from B. subtilis expressed under the thl promoter; CmR, Cm/Th resistance gene; MLSr, Em resistance gene.

FIG. 4: PCR results for confirming integration of MLSr cassette into the spo0A gene. The figure on the left illustrates the expected PCR product size for a successful chromosomal integration (lane 1), no integration (lane 2) and of λ DNA digested with BsteII ladder (lane 3). Figure on the right is a 0.7% agarose gel with EtBr detection of PCR product from chromosomal DNA of suspected integration mutants. Lanes 1 and 24 are λ DNA digested with BsteII ladder. Lanes 2-7 are PCR product from mutants obtained with no MMC exposure. Lanes 8-11 are PCR product from mutants that were exposed to 5 ng/mL MMC. Lanes 12-17 are PCR product from mutants that were exposed to 40 ng/mL MMC. Lanes 18-21 are PCR product from mutants that were exposed to 100 ng/mL MMC. Lanes 23-24 are PCR product for wild-type ATCC824 chromosomal DNA, indicative of what should be seen if integration did not occur.

FIG. 5: SKO mutant Spo0A Western Blot analysis 1. Crude protein extracts were analyzed from a time series of two different SKO mutants and compared to SKO1 and WT ATCC824. Lane details: 1—SKO mutant #1 (t=12 hrs); 2—SKO mutant #1 (t=24 hrs); 3—SKO mutant #1 (t=36 hrs); 4—SKO1 (t=18 hrs); 5—ATCC824 (t=18 hrs); 6—Invitrogen MagicMark Western protein standard (bottom to top: 20 kDa, 30 kDa, 40 kDa, 50 kDa and 60 kDa); 7—SKO mutant #2 (t=12 hrs); 8—SKO mutant #2 (t=24 hrs); 9—SKO mutant #2 (t=36 hrs); 10—Invitrogen Kaleidoscope protein standard.

FIG. 6: SKO mutant Spo0A Western Blot analysis 2. Crude protein extracts were analyzed from a time series of an SKO mutant and compared to SKO1 and WT ATCC824. Lane details: 1—SKO1 (t=18 hrs); 2—ATCC824 (t=18 hrs); 3—Invitrogen MagicMark Western protein standard (bottom to top: 20 kDa, 30 kDa, 40 kDa, 50 kDa and 60 kDa); 4—SKO mutant #3 (t=12 hrs); 5—SKO mutant #3 (t=24 hrs); 6—SKO mutant #3 (t=36 hrs); 7—Invitrogen Kaleidoscope protein standard.

FIG. 7: Two possible scenarios for single crossover events. The illustration shows what theoretically would occur if a single crossover occurred through the first or second region of homology. Additionally it illustrates what double crossover and plasmid excision events would result in.

FIG. 8: Expected product sizes from SigE integration confirmation primer set 1. Illustration of the expected product size when using primer set 1 in the SigE integration confirmation.

FIG. 9: Expected product sizes from SigE integration confirmation primer set 3. Illustration of the expected product size when using primer set 3 in the SigE integration confirmation.

FIG. 10: Expected product sizes from SigE integration confirmation primer set 4. Illustration of the expected product size when using primer set 4 in the SigE integration confirmation.

FIG. 11: PCR confirmation of SigE integration orientation. PCR results from the two SigE-KO mutants analyzed (8 and 15, mutant 3 was not obtained in this study), definitively conclude a single integration through the first region of homology because there is substantial product for primer sets 2 and 3.

FIG. 12: Composite phase contrast microscopy image of 4 of the pRecU generated mutants compared against the un-enriched plasmid control. Images were acquired from late stationary phase samples. Sporulation should be occurring or finished in cultures this old. Notice the presence of phase bright spores in the plasmid control and the 1.7% #6 mutant. There are no detectable signs of spore formation in any of the other mutant cultures.

FIG. 13. Spo0A disruption sequence. Sequencing demonstrates a perfect double crossover event in clostridia.

FIG. 14: Bacillus subtilis recU cDNA sequence (SEQ ID NO:25).

FIG. 15: SEQ ID NO:26.

FIG. 16: SEQ ID NO:27.

FIG. 17: SEQ ID NO:28.

FIG. 18: SEQ ID NO:29.

FIG. 19: SEQ ID NO:30.

FIG. 20: SEQ ID NO:31.

FIG. 21: SEQ ID NO:32.

FIG. 22: SEQ ID NO:33.

### DEFINITIONS

To facilitate an understanding of the present invention, a number of terms and phrases are defined below:

As used herein, the term “resolvase” refers to a member of a large group of site-specific recombinases, which exhibit endonuclease activity that can catalyze the intramolecular resolution reaction between heteroduplexes of recombination intermediates (e.g., cross-over structures such as Holliday-junctions). Examples of resolvases include, but are not limited to, B. subtilis ATCC23857 recU gene designated BSU22310 (SEQ ID NO:25), although other resolvases may be utilized. Additional suitable resolvase genes include, but are not limited to, those encoding Hjc (accession#Q9UWX8; SEQ ID NO:26), Endonuclease I (accession#P00641; SEq Id NO:27), RuvC (accession#P24239; SEQ ID NO:28), CceI (accession#Q03702; SEQ ID NO:29), A22R (accession#P20997; SEQ ID NO:30), RusA (accession# P40116; SEQ ID NO:31), Endonuclease VII (accession#P13340; SEQ ID NO:32), RecU (accession#P39792; SEQ ID NO:33) and homologs thereof.

As used herein, the terms “detect”, “detecting” or “detection” may describe either the general act of discovering or discerning or the specific observation of a detectably labeled composition.

As used herein, the term “gene transfer system” refers to any means of delivering a composition comprising a nucleic acid sequence to a cell or tissue. For example, gene transfer systems include, but are not limited to, vectors, microinjection of naked nucleic acid, polymer-based delivery systems (e.g., liposome-based and metallic particle-based systems) and the like.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH2PO4H2O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH2PO4H2O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH2PO4H2O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

### DETAILED DESCRIPTION OF THE INVENTION

The present invention relates to methods and compositions for engineering Clostridia species. In particular, embodiments of the present invention relate to the expression of recombinant resolvase proteins in Clostridia species.

Compositions and methods of embodiments of the present invention find use in recombinant protein expression of resolvase proteins in any Clostridia species or other prokaryotes without autologous expression of a resolvase or other suitable species. Embodiments of the invention also apply to overexpression of autologous or heterologous resolvases in an organism that contains a resolvase, whereby the overexpression enhances the genomic integration capability and the plasticity of the genome by homologous recombination. The resolvase to be used include, but are not limited to, an existing resolvase from any organism or a protein-engineered or synthetic resolvase, which might have improved or different protein suitable for a specific organism or application.

Embodiments of the present invention provide a new approach for genetically altering Clostridia. Certain embodiments of the present invention provide recombinant expression of a resolvase protein in any Clostridia species. Resolvases are a well-known class of proteins that perform a defined role in Holliday junction resolution during homologous recombination (Lilley, D. M. and M. F. White, Nat Rev Mol Cell Biol, 2001. 2(6): p. 433-43). There are a number of distinct resolvase enzymes, and resolvase activity is ubiquitous to nearly all bacteria (Lilley and White, supra; Rocha et al., PLoS Genet, 2005. 1(2): p. e15). However, comparative genomics analyses indicate that Clostridia are a rare class of bacteria that do not contain genes for any recognizable resolvase protein (Rocha et al., supra). There is no experimental evidence to contradict such a conclusion, and a wealth of experimental data support it. For example, induced homologous recombination for the purpose of generating gene disruptions is an infrequent event in Clostridia (Heap, J. T., et al., J Microbiol Methods, 2007. 70(3): p. 452-64), which is due to a lack of resolvase activity.

In some embodiments, resolvase activity is re-introduced to Clostridia or other bacteria lacking resolvase systems via the recombinant expression of a resolvase protein.

One utility of the technology described herein is the enhanced capability to genetically modify Clostridia. This is demonstrated through the proceeding diverse examples, which are: 1) site-specific homologous recombination for perfect double crossover gene disruption, 2) site-specific homologous recombination for single crossover gene disruption, 3) site-specific homologous recombination for single or double crossover gene knock-in, and 4) inducing genetic heterogeneity through resolvase induced chromosomal recombination and/or mutation events.

### I. Homologous Recombination

### Homologous Recombination

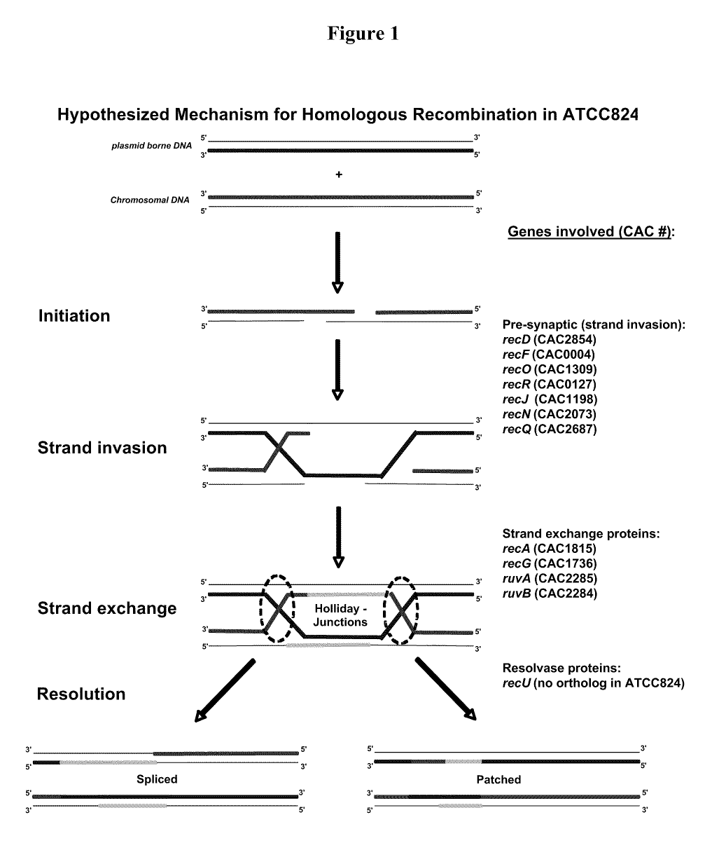

Homologous recombination is a housekeeping process involved in the maintenance of chromosome integrity and generation of genetic variability that is nearly ubiquitous to all microorganisms (Rocha et al., supra; Fraser et al., Science, 2007. 315(5811): p. 476-80; Lorenz et al., Microbiol Rev, 1994. 58(3): p. 563-602). The cellular machinery involved is not necessarily conserved, but the general series of events is common to all microorganisms studied to date. The typical series of events for homologous recombination are initiation, strand-invasion, strand-exchange, and Holliday junction resolution (Rocha et al., surpa; Hiom, Curr Biol, 2000. 10(10): p. R359-61; Kowalczykowski, Trends Biochem Sci, 2000. 25(4): p. 156-65), see FIG. 1. Within specific genera of bacteria, the proteins involved in homologous recombination are fairly well conserved and are given for Clostridia in FIG. 1. The specific C. acetobutylicum genes involved are given in Table 1 and FIG. 1, which were determined by a best-best blast search to Bacillus subtilis ATCC23857. B. subtilis serves as the model Gram-positive organism.

### Genetic Manipulation Via Homologous Recombination

Homologous recombination is routinely employed in molecular biology for a multitude of applications such as inserting recombinant genes into a host chromosome, targeting host genes for inactivation, and engineering host-reporter fusion proteins. More elegant genetic manipulation approaches employ homologous recombination to accelerate horizontal gene transfer (also known as lateral gene transfer) (Frost et al., Nat Rev Microbiol, 2005. 3(9): p. 722-32; Gogarten and Townsend, Nat Rev Microbiol, 2005. 3(9): p. 679-87; Smets and Barkay, Nat Rev Microbiol, 2005. 3(9): p. 675-8; Sorensen et al., Nat Rev Microbiol, 2005. 3(9): p. 700-10; Thomas and Nielsen, Nat Rev Microbiol, 2005. 3(9): p. 711-21).

Horizontal gene transfer refers to the phenomenon of genetic material transfer from one cell to another cell that is not its offspring. Additionally, homologous recombination can also be utilized to generate random genetic variability compared to the wild type, which can subsequently be screened and analyzed for novel, desirable cellular phenotypes.

### Significance of Resolvases

As mentioned previously, resolvases are well-characterized proteins involved in the resolution stage of homologous recombination and generically in DNA repair (Rocha et al., supra; Hiom, supra; Kowalczykowski, supra). More specifically they are the essential enzymes involved in Holliday junction resolution (Biertumpfel et al., Nature, 2007. 449(7162): p. 616-20; Hadden et al., Nature, 2007. 449(7162): p. 621-4; Kelly et al., Proteins, 2007. 68(4): p. 961-71; Webb et al., J Biol Chem, 2007. 282(47): p. 34401-11). Holliday junctions are four way DNA intermediate complexes witnessed during homologous recombination (Duckett et al., Cell, 1988. 55(1): p. 79-89). Resolvases are diverse and not necessarily conserved between different classes of bacteria, but they are ubiquitous to nearly all bacteria and archaea (Lilley and White, supra). There are two majority resolvases found natively in the genomes of Gram-negative and Gram-positive bacteria. These are ruvC and recU, respectively (Rocha et al., supra; Fernandez et al., J Bacteriol, 1998. 180(13): p. 3405-9). The significance of resolvases, and more specifically recU in Gram-positive organisms was studied via deletion mutants and tested by the deficiency in DNA repair and intramolecular recombination (Fernandez et al., supra; Carrasco et al., Nucleic Acids Res, 2005. 33(12): p. 3942-52; Carrasco et al., J Bacteriol, 2004. 186(17): p. 5557-66). These studies indicate that RecU is involved in Holliday junction resolution for Gram-positive organisms. Subsequent studies determined high-resolution structures of RecU from Bacillus subtilis and Bacillus stearothermophilus and proposed detailed models for how the RecU protein physically interacts with the Holliday junction (Kelly et al., Proteins, 2007. 68(4): p. 961-71; McGregor, et al., Structure, 2005. 13(9): p. 1341-51).

### Absense of Resolvases in Clostridia

A recent comparative genomics study of the essential homologous recombination machinery from 110 bacterial species demonstrated that Clostridia were void of any obvious resolvase gene (Rocha et al., supra). Homology searches were performed against B. subtilis, and assigned if a protein was the bidirectional best hit with at least 40% similarity in DNA sequence and less than 30% difference in length. More specifically the authors demonstrate that all of the three Clostridia genomes analyzed were void of a B. subtilis recU homolog (C. acetobutylicum GenBank#AE001347 and AE0013478, RefSeq#NC_{—}003030 and NC_{—}001988; C. perfringens GenBank#AP003515 and BA000016, RefSeq#NC_{—}003366 and NC_{—}0030242; C. tetani, GenBank#AE015927, RefSeq#NC_{—}004557 and NC_{—}004565). The specific recU gene sequence was BSU22310 from B. subtilis ATCC23857 (GenBank #AL009126, Refseq NC_{—}000964). Only ten genomes out of the 110 appeared to be resolvase deficient. Of the remaining seven resolvase deficient genomes, at least 4 appeared to be completely void of any sort of recombination system. No other division of bacteria appeared to have all the essential recombination proteins except for one, and especially not a protein that is as ubiquitous as a resolvase.

To further this analysis, six additional genomes (three fully sequenced/annotated and 3 draft sequences) of well known pathogenic, solvent forming, or pharmaceutically relevant Clostridia species were investigated. These genomes were C. difficile 630 GenBank#AM180355 and AM180356, RefSeq#NC_{—}009089 and NC_{—}008226; C. novyi NT GenBank#CP000382, RefSeq#NC_{—}008593; C. thermocellum GenBank#CP000568, RefSeq#NC_{—}009012; C. beijerincki draft sequence; C. cellulolyticum draft sequence; C. phytofermentas ISDg draft sequence. Of these additional six species, five were void of a resolvase gene. C. phytofermentas ISDg was the only genome with a homologous recU gene. Results are given in Table 2.

Additionally, there are numerous reports of the difficulty in obtaining gene disruptions in any Clostridia species via homologous recombination. In a recent paper (Heap et al., J Microbiol Methods, 2007. 70(3): p. 452-64) describing a different approach to gene disruptions in Clostridia, the authors clearly state the difficulty in generating gene disruptions via native homologous recombination. In spite of many concerted attempts there have been only a handful of gene disruptions accomplished in relatively few Clostridium species, specifically C. acetobutylicum, C. perfringens, C. difficile and C. beijerinckii (Desai and Papoutsakis, Appl Environ Microbiol, 1999. 65(3): p. 936-45; Green and Bennett, Appl Biochem Biotechnol, 1996. 57-58: p. 213-221; Green et al., Microbiology, 1996. 142 (Pt 8): p. 2079-86; Harris et al., J Bacteriol, 2002. 184(13): p. 3586-97; Varga et al., J Bacteriol, 2004. 186(16): p. 5221-9. Gene disruptions have also been reported in C. tyrobutyricum (Liu et al., Biotechnol Prog, 2006. 22(5): p. 1265-75; Zhu et al., Biotechnol Bioeng, 2005. 90(2): p. 154-66. Overall, the efficiency of chromosomal integration is very low in all species and the method of integration is typically non-ideal and/or unstable.

### Targeted Gene Disruptions in Clostridia

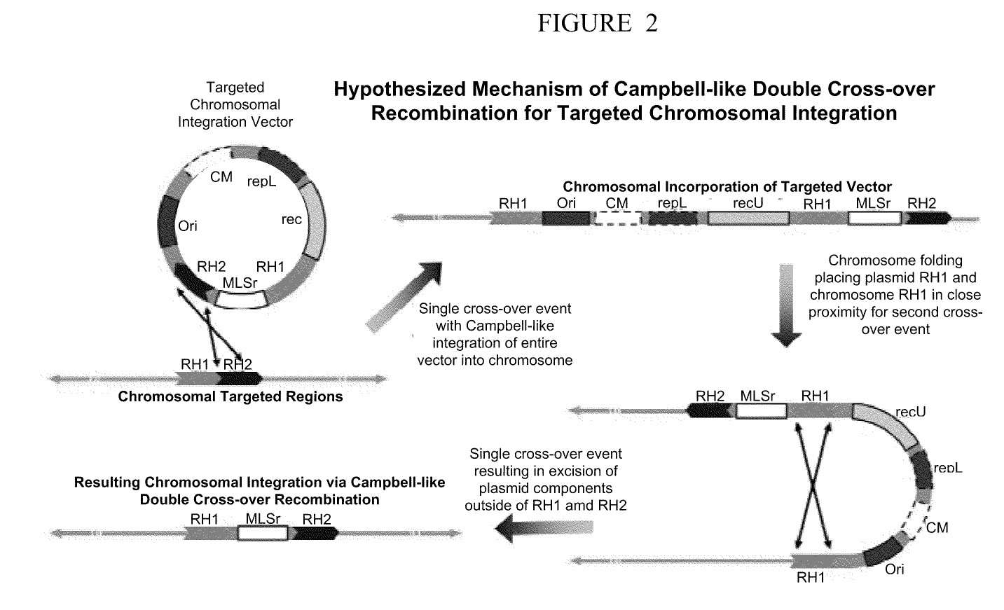

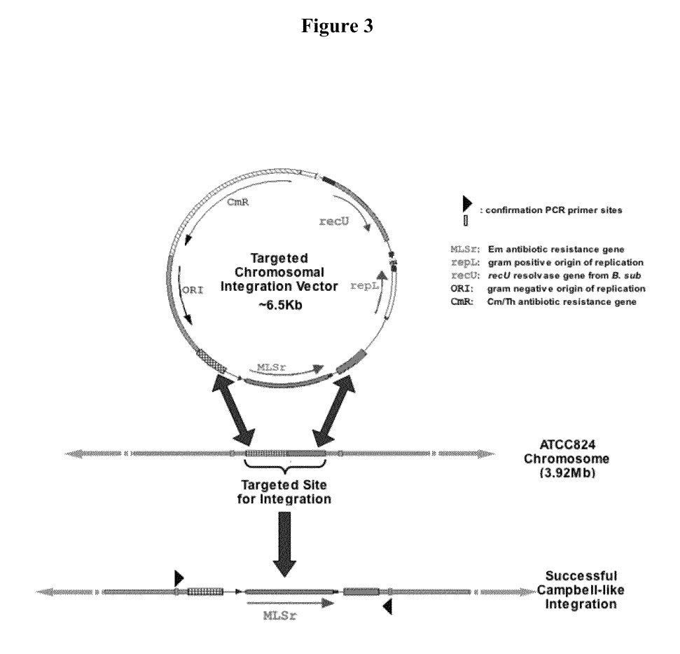

In regards to gene disruption via homologous recombination, the current state of the art is to employ the Clostridia host's homologous recombination machinery for double crossover recombination. Recombination occurs between parent chromosome and plasmid-borne homologous regions that flank a selectable marker, see FIGS. 1-3.

For C. acetobutylicum there are only three published reports of site-specific integration; two via non-replicating (suicide) plasmids (Green and Bennett, Appl Biochem Biotechnol, 1996. 57-58: p. 213-221; Green et al., Microbiology, 1996. 142 (Pt 8): p. 2079-86) and one via a replicating plasmid (Harris et al., supra). The first attempt utilized a suicide plasmid with an integration cassette composed of ˜225 bp nucleotide sequences of contiguous homology flanking a macrolide-lincosamide-streptogramin B resistance (MLSr) gene. The MLSr gene confers erythromycin (EM) resistance. The plasmid was introduced into C. acetobutylicum via electroporation, and knockout mutants were selected for EM resistance. Only mutants that had undergone a recombination event could maintain EM resistance. This technique was successful three times for the generation of pta, bk and aad mutants (Green and Bennett, Appl Biochem Biotechnol, 1996. 57-58: p. 213-221; Green et al., Microbiology, 1996. 142 (Pt 8): p. 2079-86). However, integration efficiency was very low, 0.5 mutants/μg transformed KO plasmid DNA, and unsuccessful for many additional targets.

A second approach was later developed that employed a replicating plasmid. When using the replicating approach, an additional selection marker is required outside the integration cassette in order to prove the loss of plasmid after a successful of double crossover recombination. For this approach a thiamphenicol (CM/TH) resistance gene was employed. Following electroporation, transformed cells are selected for EM resistance. Transformants were then vegetatively transferred six times on non-antibiotic nutrient plates. The seventh and eighth transfers were onto EM and TH containing plates, respectively. These two plates were compared for regions of growth on EM but not on TH, suggesting double crossover recombination and loss of plasmid. So far this approach has been successful at generating only a handful of mutants such as spo0A mutant (Harris et al., supra), CAC8241 mutant and ctfAB mutant, and all subsequent attempts at additional targets have been unsuccessful.

Among other Clostridia species there have been few successful attempts at generating targeted chromosomal integrations via suicide and replicating plasmids (Huang et al., FEMS Microbiol Lett, 2004. 233(2): p. 233-40; Sarker et al., Mol Microbiol, 1999. 33(5): p. 946-58; Raju et al., BMC Microbiol, 2006. 6: p. 50). Thus, a different sort of gene disruption system was adapted to Clostridia in order to increase site-specific integration efficiency. The group II intron system developed by the Lambowitz lab at University of Texas-Austin, now commercialized by Sigma-Aldrich (TargeTron™), has been employed on multiple occasions to generate gene disruptions in C. perfringens and C. acetobutylicum (Chen et al., Plasmid, 2007. 58(2): p. 182-9; Chen et al., Appl Environ Microbiol, 2005. 71(11): p. 7542-7; Shao et al., Cell Res, 2007. 17(11): p. 963-5; Wei et al., Cancer Lett, 2008. 259(1): p. 16-27). A more intensive study modified the TargeTron™ specifically for application in Clostridia species. The ClosTron system has been employed to generate gene disruptions in C. acetobutylicum, C. difficile, C. botulinum and C. sporogenes (Heap et al., supra). Group II introns are naturally occurring autocatalytic retrotransposable elements that include a six stem-loop RNA structure complexed with an intron-encoded protein (IEP). The IEP exhibits four unique activities: 1) maturase for intron splicing, 2) DNA binding for target site recognition, 3) endonuclease for nicking host chromosome and 4) reverse transcriptase for forming intron cDNA. Group II introns can insert RNA directly into target DNA sequences and then reverse transcribe themselves. DNA is targeted mainly by base pairing of the intron RNA, however the IEP also recognizes a few base pairs. Subsequently, group II introns can theoretically be engineered to target any desired DNA sequence by modifying the intron RNA (Karberg et al., Nat Biotechnol, 2001. 19(12): p. 1162-7).

### Generating Genetic Variation in Clostridia

Induced genetic variation at the genome scale, coupled with fitness selection, is a popular approach for accelerating the development of new, improved bacterial strains. Some of these techniques include the screening of chemically mutated populations, interference RNA libraries, transposon mediated mutant libraries, and recombinant DNA plasmid libraries. The screening of chemically mutated populations, recombinant DNA plasmid libraries and transposon mediated mutant libraries has been performed in C. acetobutylicum with some success (Annous and Blaschek, Appl Environ Microbiol, 1991. 57(9): p. 2544-8; Babb et al., FEMS Microbiol Lett, 1993. 114(3): p. 343-8; Borden and Papoutsakis, Appl Environ Microbiol, 2007. 73(9): p. 3061-8; Bowring and Morris, J Appl Bacteriol, 1985. 58(6): p. 577-84; Rogers and Palosaari, Appl Environ Microbiol, 1987. 53(12): p. 2761-2766). However, these approaches all have considerable drawbacks. Chemical mutagenesis is limited by a lack of easily selectable markers, thus new phenotypes are only discovered via obvious phenotypic changes. Recombinant DNA libraries are limited to an individual genetic modification, which also limits the subsequent state space to the constraints of previous modifications. Interference RNA libraries are hampered by incomplete silencing of targets, and transposon mediated mutation is limited by availability of genetic systems within a given host.

### II. Expression of Resolvases in Clostridia Species

In some embodiments, the present invention provides compositions and methods for expressing a resolvase protein in any Clostridia species. Some embodiments utilize either a plasmid borne copy or a chromosomal integration copy of the resolvase gene in vivo.

### Resolvase Cassette

The present invention is not limited to a particular resolvase enzyme. Any suitable resolvase enzyme may be utilized. In some embodiments, the resolvase is the B. subtilis ATCC23857 recU gene designated BSU22310 (SEQ ID NO:25), although other resolvases may be utilized. Additional suitable resolvase genes include, but are not limited to, those encoding Hjc (accession#Q9UWX8; SEQ ID NO:26), Endonuclease I (accession#P00641; SEq Id NO:27), RuvC (accession#P24239; SEQ ID NO:28), Cce1 (accession#Q03702; SEQ ID NO:29), A22R (accession#P20997; SEQ ID NO:30), RusA (accession#P40116; SEQ ID NO:31), Endonuclease VII (accession#P13340; SEQ ID NO:32), RecU (accession#P39792; SEQ ID NO:33) and homologs thereof (See e.g., NCNI curated Prokaryotic Protein Clustering database (Klimke et al., 2009. The National Center for Biotechnology Information's Protein Clusters Database. Nucleic acids research 37:D216-D223)).

In some embodiments, the expression of the resolvase gene is placed under the strong, native thiolase transcription promoter (thL) from C. acetobutylicum ATCC824, although other promoters may be used. In some embodiments, transcription termination is ensured by a rho independent terminator downstream of the recU gene, although other transcription terminators may be used. The combination of promoter, resolvase gene and rho independent terminator is referred to as a resolvase cassette. Other suitable promoters include, but are not limited to, other native Clostridia promoters such as the C. acetobutylicum phosphotransbutyrylase (ptb) promoter, C. acetobutylicum acetoacetate-decarboxylate (adc) promoter, C. thermocellum endogluconase A (celA) promoter, C. pasteurianum ferredoxin promoter, and non-native Clostridia promoters such as the “fac” promoter. Other suitable terminator sequences include, but are limited to, any suitable 7-24 basepair sequence that upon transcription can form a thermodynamically stable stem-loop structure capable of causing intrinsic transcription termination.

For double and single crossover gene disruption, as well as gene knock-ins, the resolvase cassette is incorporated into a similar replicating plasmid to that described above.

For inducing genetic heterogeneity through resolvase induced chromosomal recombination and/or mutation events, several approaches are utilized. In some embodiments, the resolvase cassette is expressed from a plasmid in C. acetobutylicum. The RecU protein is constantly generated, thus improving functionality of the recombination system, and encouraging random recombination events within the genome of the host cell. Next, cells are stressed to sub-lethal stress conditions or non-optimal growth conditions in general and random mutations are allowed to accumulate over time. Subsequently this results in a pool of genetically heterogeneous cells, each with varying phenotypes that can be screened for desirable traits. In the second approach, the resolvase cassette is integrated into the genome and then stressing and screening are performed.

The present invention is not limited to the expression of resolvase activity in Clostridia or any particular application. The technology is not limited to any specific application, rather the utility of resolvase expression in Clostridia or other organisms in general.

In some embodiments, the present invention provides kits for use in engineering bacteria such as Clostridia species. The kit may include any and all components necessary, useful or sufficient for engineering and screening bacteria including, but not limited to, the resolvase cassettes, buffers, control reagents (e.g., bacterial samples, positive and negative control sample, etc.), reagents for screening for positive clones, reagents for stressing cells, labels, written and/or pictorial instructions and product information, inhibitors, labeling and/or detection reagents, package environmental controls (e.g., ice, desiccants, etc.), and the like. In some embodiments, the kits provide a sub-set of the required components, wherein it is expected that the user will supply the remaining components. In some embodiments, the kits comprise two or more separate containers wherein each container houses a subset of the components to be delivered.

### III. Uses

The compositions and methods described herein find use in a variety of applications including, but not limited to, the genetic modification of Clostridia and other species lacking native resolvase proteins.

In some embodiments, the compositions and methods for genetic modification of Clostridia described herein find use in the disruption of specific genes, including gene knock-in and knock-outs.

In other embodiments, the compositions and methods for genetic modification of Clostridia described herein find use in screening altered populations for improved properties. For example, in some embodiments, chemically mutated populations, interference RNA libraries, transposon mediated mutant libraries, and recombinant DNA plasmid libraries are screened. In some embodiments, the compositions and methods of the present invention are used to insert reporter genes (e.g., antibiotic resistance genes) into Clostridia species (e.g., to aid in the screening of altered populations).

In some embodiments, an exogenous resolvase gene (e.g., on a plasmid or integrated into a genome) is used to promote recombination and mutation under selective conditions (e.g., the presence of butanol).

Clostridia and other bacterial species engineered or screened using the compositions and methods of the present invention find use in a variety of industrial, medical, and research applications. Examples include, but are not limited to: 1) fermentative production of chemical feedstocks for subsequent synthesis into acrylate/methacrylate esters, glycol ethers, butyl-acetate, amino resins and butylamines; 2) fermentative conversion of biodiesel glycerol waste streams to propionic acids; 3) fermentative production of acetone, ethanol and/or butanol production as bulk chemicals; 4) fermentative production of butanol and/or ethanol as a transportation fuel (biofuel); 5) fermentative production of all aforementioned chemical species from renewable resources such as cellulosic and hemicellulosic materials; 6) engineering better Clostridial-directed enzyme prodrug therapies as alternatives to chemotherapeutics; 7) basic research applications; and 8) Bioremediation.

### EXPERIMENTAL

The following examples are provided in order to demonstrate and further illustrate certain preferred embodiments and aspects of the present invention and are not to be construed as limiting the scope thereof.

### Example 1

### Construction of Resolvase Cassette

The resolvase cassette was constructed by cloning the recU (BSU22310) open reading frame (ORF) plus native Shine-Dalgarno (SDG) sequence from B. subtilis ATCC23857 (GenBank #AL009126, Refseq NC_{—}000964) into pSOS95del (Tummala et al., 2003. J. Bacteriol. 185:1923-1934)) via a directional sticky end ligation of BamHI and KasI. The recU and engineered BamHI and KasI digest sites were amplified from B. subtilis ATCC23857 genomic DNA with the recU-F and recU-R primer set. The 719 bp PCR product was purified, double digested with BamHI and KasI, and phosphorylated. pSOS95del was generating by double digesting pSOS95 with BamHI and KasI, gel band purifying the 4979 bp plasmid backbone, and dephosphorylating. The pSOS95del plasmid backbone and recU PCR product were ligated via New BioLabs® (NEB) Quick Ligase and cloned into Invitrogen® One Shot® TOP10 E. coli. The resulting plasmid we call pRecU. The resolvase cassette was PCR amplified out of pRecU with the recU-cass-F and recU-cass-R primer set and NEB Vent polymerase for blunt end product.

### Example 2

### spo0A Gene Disruption Mutant—First Ever-Perfect Double Crossover Mutant in any Clostridia Species

Construction of spo0A Targeted Gene Disruption Plasmid

For the C. acetobutylicum spo0A gene (CAC2071) targeted plasmid, the resolvase cassette was incorporated into the pETSPO[25] plasmid. The pETSPO plasmid was linearized with the blunt end cutting SmaI endonuclease and dephosphorylated. The resolvase cassette was ligated into the linear pETSPO plasmid via NEB Quick Ligase reaction and cloned into Invitrogen® One Shot® TOP10 E. coli. The final replicating, spo0A targeted plasmid is called pKORSPO0A.

Generation of spo0A Disruption Mutants

Targeted gene disruption plasmid was transformed into C. acetobutylicum via a previously reported electroporation protocol (Mermelstein et al., Biotechnology (N.Y.), 1992. 10(2): p. 190-5). Prior to transforming, plasmid DNA must be site specifically methylated to avoid degradation by the clostridial endonuclease CAC8241. Plasmid DNA was methylated by shuttling through E. coli ER2275 pAN2. pAN2 contains a gene encoding for the site-specific methyltransferase.

Transformants were vegetatively transferred every 24 hrs for 5 days via replica plating on solid 2×YTG plates supplemented with the antibiotic disrupting the gene of interest. For pKORSPO0A an erythromycin (EM) antibiotic marker is disrupting the spo0A gene and a TH marker is on the backbone of the plasmid. Vegetative transfers were performed under EM selection. Antibiotic concentrations were 40 μg/mL for EM and 20 μg/mL for TH. After five days, the cells were again vegetatively transferred for an additional five days under no antibiotic selection. This is performed for plasmid curing (to lose the plasmid). After five days of curing, the cells were transferred to plates containing the antibiotic disrupting the gene of interest, and allowed to grow for 24 hrs. These plates were then transferred to plates supplemented with the antibiotic on the vector backbone, allowed to grow for 24 hrs and compared to the previous plates. Areas of growth and no growth on the plates supplemented with the antibiotic disrupting the gene of interest and antibiotic on the vector backbone, respectively, were indicative of chromosomal integrations and more specifically double crossover events. These putative gene disruptions were streaked on plates supplemented with the antibiotic disrupting the gene of interest, allowed to grow for 24 hrs, and then replica plated onto the other antibiotic plate in order to clearly demonstrate antibiotic sensitivity.

### Confirming Gene Disruption Mutants

Gene disruption mutants were confirmed via DNA sequencing. Genomic DNA was prepared from the mutants via a modified phenol:chloroform:isoamyl alcohol extraction with ethanol precipitation and stored at 4° C. in TE buffer (Mermelstein et al., supra). Sequencing primers were designed such that they amplified off of flanking regions of the chromosome where the gene disruption should have occurred but would not have been affected by the integration. Additional primers were designed within the region of disruption allowing for sequencing out of the antibiotic marker and into the chromosome because sequence read lengths were not always sufficient for confirming the exact orientation of integration. Sequencing primers are given in Table 5.

Western Blot Confirmation of spo0A Mutant

Western Blot analysis was performed on 10 μgs of protein crude extract from disruption mutants. The spo0A primary antibody was an affinity purified polyclonal antibody. Crude extracts from mutants were compared to wild-type (WT) and a previously generated spo0A disruption mutant (Harris et al., supra).

Results from spo0A Disruption Mutants

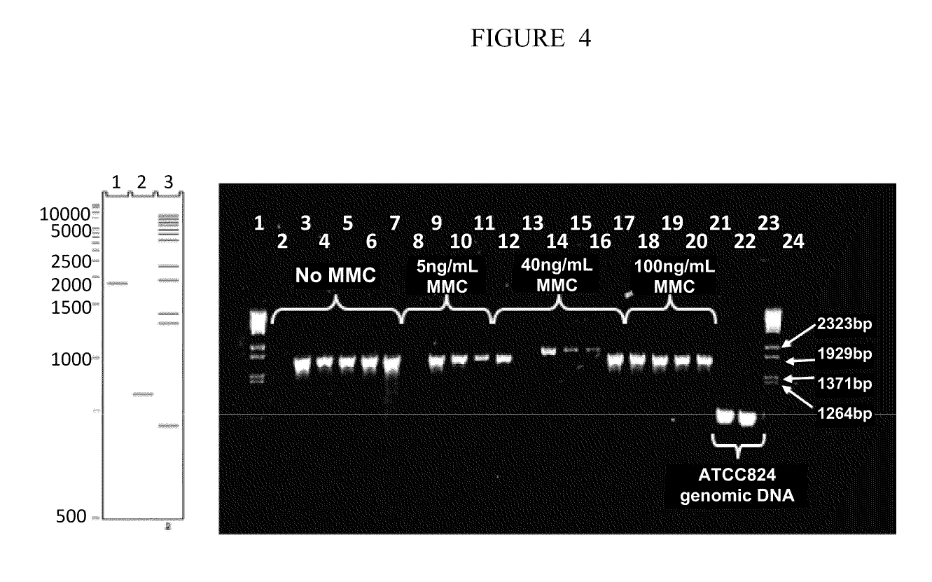

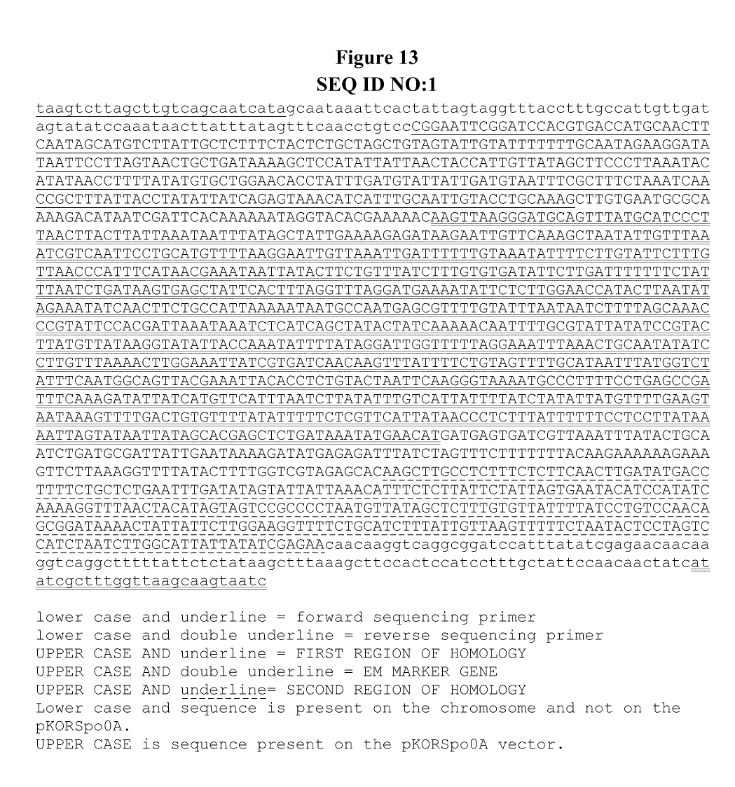

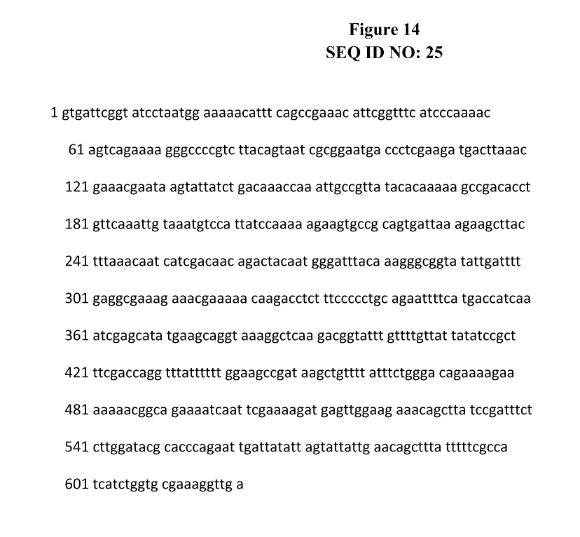

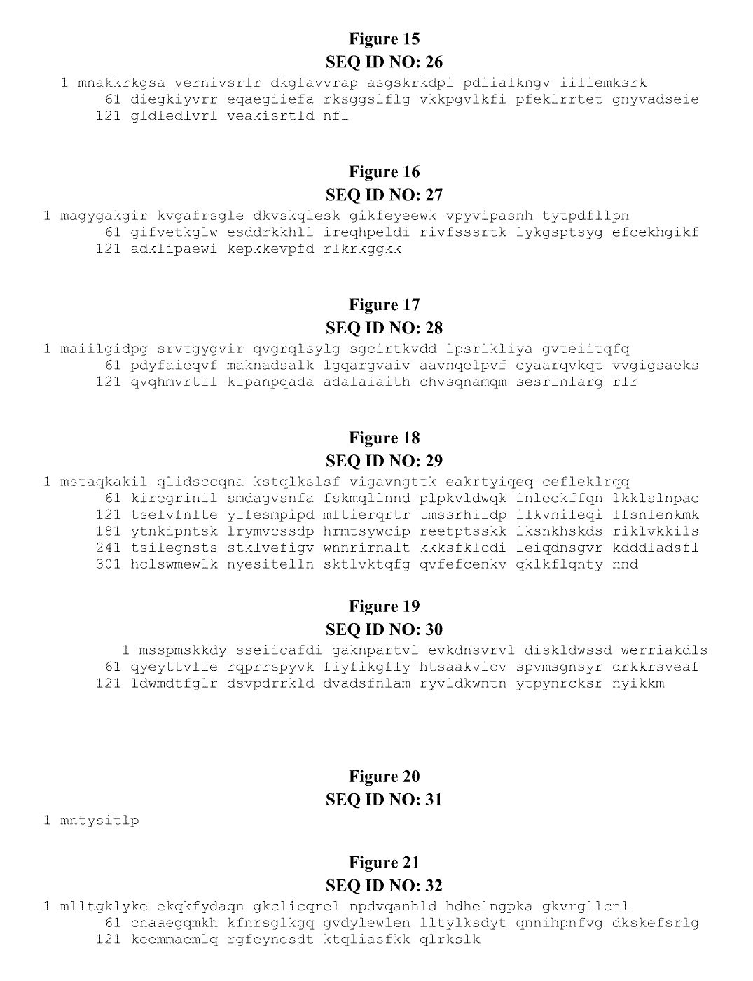

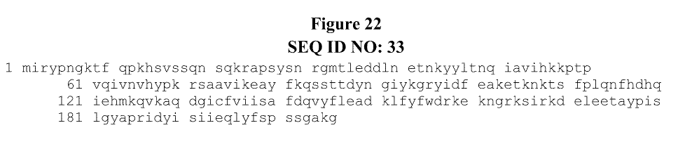

Over 20 regions of growth on the final EM plate did not grow on the TH plate. Cells from these regions were streaked onto fresh EM plates, grown for 24 hrs and then replica plated onto TH plates. None of the re-streaked cells grew on TH plates. Identical vegetative transfer experiments with the addition of the DNA mutating agent mitomycin C (MMC) at three different concentrations were performed. Results were very similar and conclusive that MMC is not needed. Genomic DNA was isolated from 20 of the mutants, and a confirmation PCR was performed, refer to FIG. 4. The mutants are referred to as SKO mutants. For 17 of the 20 PCR reactions, a product band indicative of a double crossover event was obtained. The other 3 did not yield product. Two WT controls, which generated product indicative of no integration event, were also performed. PCR product was sequenced and the results confirmed perfect double crossover events for all mutants. Sequencing primers were Spo0A-KO-conf-F/R. This is the first time that perfect Campbell-like double crossover gene disruption mutants have ever been reported in any Clostridia species. Sequencing results are shown in FIG. 13.

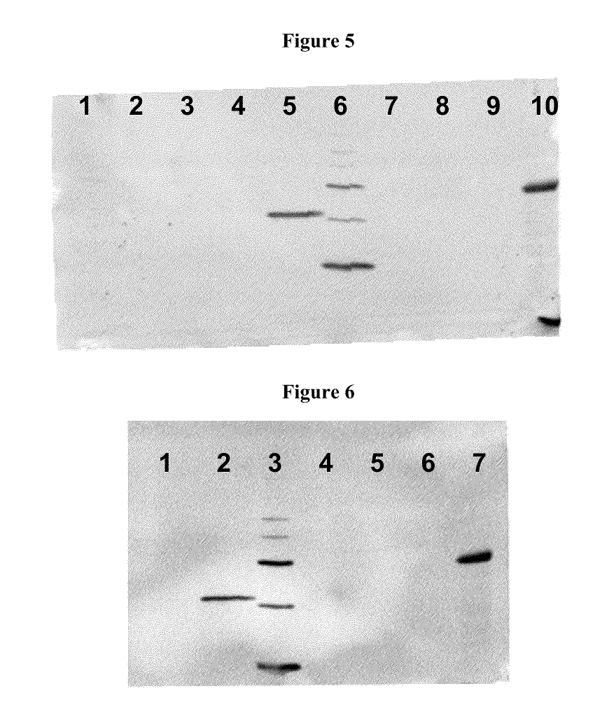

Western Blot analysis was performed on crude extracts from the gene disruption mutants. Crude extracts from three of mutants were compared to WT and a previously reported spo0A disruption mutant crude extracts. Samples for the mutants were taken at multiple time points, during which Spo0A expression is known to occur. Results clearly show the complete absence of any Spo0A in the mutant crude extracts as well as the previously reported mutant SKO1, refer to FIGS. 5 & 6. There is a distinct single band for the WT at about 32 kDa in size, just as expected.

Phase contrast microscopy was performed on the mutants that Western Blot analysis was performed on. Results were identical to the previously reported results of an asporogenous phenotype (Harris et al., supra).

### Example 3

### sigE Gene Disruption Mutant—Single Crossover Disruption Mutant

Construction of sigE Targeted Gene Disruption Plasmid

For the C. acetobutylicum sigE gene (CAC1695) targeted plasmid, the disrupted sigE gene fragment was constructed in the pCR8-GW-TOPOTA™ cloning plasmid from Invitrogen®. A 559 bp region of the sigE gene was PCR amplified with Taq polymerase and SigE-F/R primer set, and then cloned into the pCR8-GW-TOPOTA™ cloning plasmid and One Shot® TOP10 E. coli via manufacturer suggestions. The resulting plasmid is called pCR8-SigE. The sigE gene fragment was then disrupted in approximately the middle of the gene fragment via a NdeI endonuclease digestion. The linear plasmid was blunt ended via NEB® Klenow (large fragment) treatment and then dephosphorylated. An antibiotic cassette was cloned into the linear plasmid via NEB Quick Ligase and cloned into Invitrogen® One Shot® TOP10 E. coli. The antibiotic cassette for the sigE disruption was a modified chloramphenicol/thiamphenicol (CM/TH) marker. The resulting plasmid is designated pCR8-SigE/CM/ptB. The SigE/CM/ptB gene disruption cassette was PCR amplified out of pCR8-SigE/CM/ptB with the SigE-F/R primer set and Vent polymerase for blunt end product. The replicating plasmid backbone with the resolvase cassette was prepared by double digesting pRecU with AvaII and XcmI, and gel band purifying the resulting 4398 bp product. This plasmid backbone was blunt ended via NEB® Klenow (large fragment) treatment and then dephosphorylated. The 1610 bp SigE/CM/ptB gene disruption cassette was ligated into the pRecU backbone via NEB Quick Ligase and cloned into Invitrogen® One Shot® TOP10 E. coli. The final replicating, sigE targeted plasmid is called pKORSIGE.

### Construction of the Modified TH Marker

A new CM/TH antibiotic marker was constructed, which replaced the old SDG with an optimal SDG and placed its expression under the transcriptional control of either the thL or phosphotransbutyrylase promoter (ptB). A 1567 bp region was PCR amplified from pLHKO with CM-F and CM-R primers. This region contains the annotated CM/TH marker, including the associated promoter and terminator regions. This serves as the unmodified antibiotic marker. A 687 bp modified CM/TH marker was generated from the 1567 bp region by PCR with mod-CM/SDG-F and mod-CM/SDG-R primers. The CM/TH modified marker includes the following: the 624 bp ORF, a newly designed Shine-Delgarno sequence (SDG), a 5′-BamHI restriction site and a 3′-KasI restriction site. The mod-CM/SDG-F primer included 33 bps of homology to the original CM/TH marker, including the ATG start codon, 6 additional codons, and 12 bps upstream of the start codon. It also included 23 bps of new sequence on the 5′-end of the primer that coded for a new “more conserved” SDG and a BamHI restriction site. The mod-CM/SDG-R primer consisted of 21 bps of homology to the CM/TH marker, specifically the last by of the ORF and 20 additional non-coding bps of homology, and 7 new nucleotides on the primer 5′-end encoding a KasI restriction site. The resulting PCR product was double digested with BamHI and KasI and directionally cloned into either pSOS94del or pSOS95del, for ptB or thL promotion respectively. pSOS95del was generated as described in “Construction of resolvase cassette,” and the pSOS94del is the exact same plasmid backbone but with the ptB promoter instead of thL. The modified antibiotic cassettes were then PCR amplified out of the resulting p95CM and p94CM plasmids with the recU-F/R primer set.

Generation of sigE Disruption Mutants

An identical protocol to that employed for generating the spo0A mutants was used for generating the sigE disruption mutants. However the antibiotics were reversed, meaning the TH marker was disrupting the sigE gene fragment and the EM marker was on the vector backbone. Thus the replica plating began with TH selectivity instead of EM, and TH resistance and EM sensitivity indicated putative integration mutants.

### Confirming Gene Disruption Mutants

Gene disruption mutants were confirmed in identical fashion to that of spo0A mutants. Primers for sequencing are given in Table 5.

Results from sigE Disruption Mutants

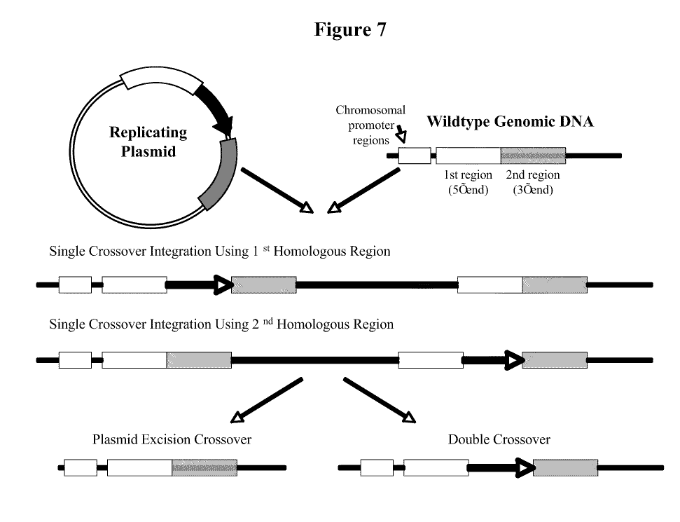

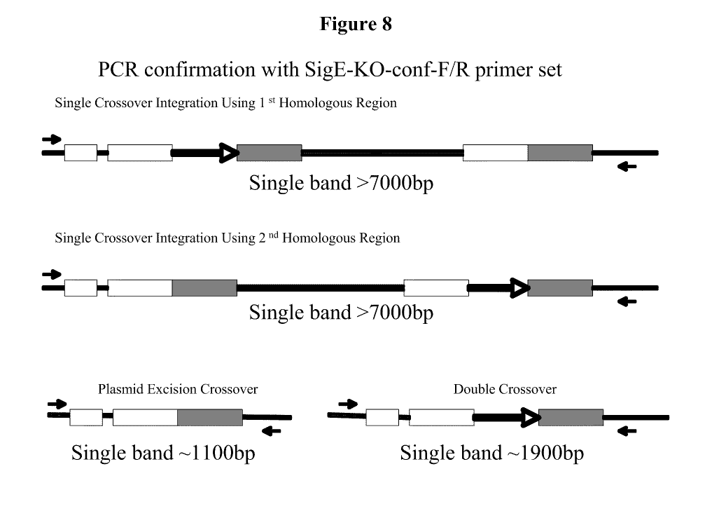

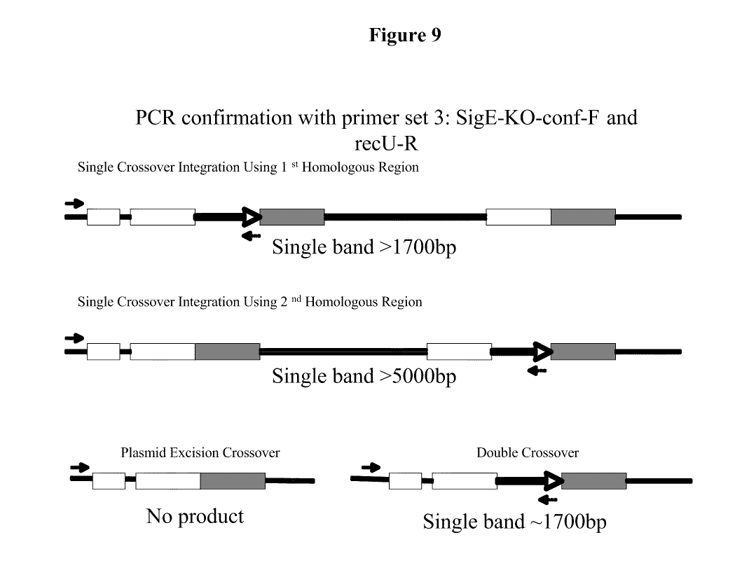

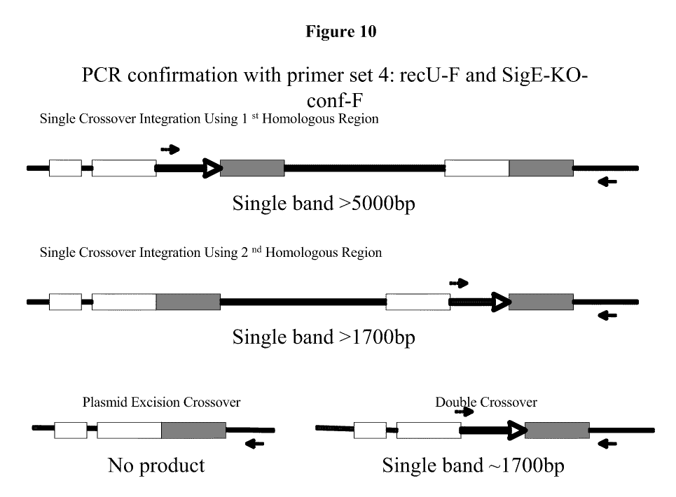

Numerous putative gene disruption mutants resolved on the final TH plating following the complete replica plating protocol. These mutants were identified by comparing to the EM plate after 24 hrs of growth. However, the majority of these regions on the EM plate actually showed growth after 72 hrs of incubation. The explanation, as the results indicate, is that a single crossover gene disruption event took place. In the case of a single crossover event, the entire plasmid gets incorporated into the chromosome and its orientation is dependent on which region of homology underwent crossover. Therefore both antibiotic markers were incorporated into the chromosome. However, since the EM marker was not under the control of a strong Clostridia promoter and present as only a single copy (plasmid was lost by this time), it took longer than 24 hrs for strains harboring a single chromosomal copy of the EM gene to grow on EM plates. PCR confirmation of gene disruption was performed for two of these mutants. Results indicated that the first region of homology (5′-end of the sigE gene) had performed the crossover, which effectively disrupted any full copy of the sigE gene. If a single crossover occurred and the entire plasmid incorporated, the confirmation PCR would result in a PCR product ˜7000 bp large. Primer sets that theoretically could only amplify PCR product if the plasmid had incorporated into the chromosome at the desired location used. Specifically, the following primer sets were used: 1) SigE-KO-conf-F and SigE-KO-conf-R; 2) recU-F and recU-R; 3) SigE-KO-conf-F and recU-R; and 4) SigE-KO-conf-R and recU-F. Refer to FIGS. 7-11 for a schematic explanation and PCR results.

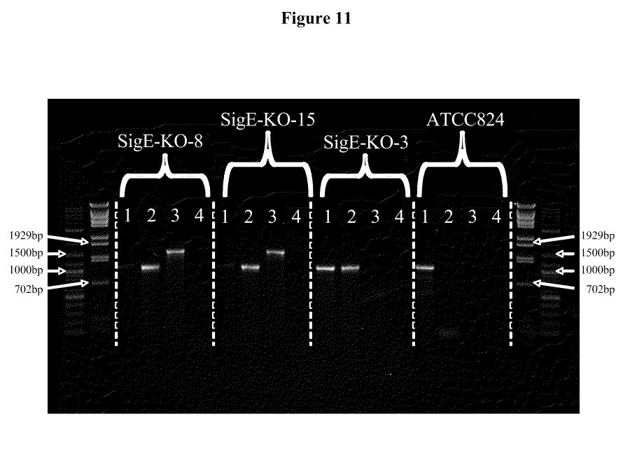

In the case of no integration, one should witness an intense ˜1000 bp PCR product band for primer set 1 when running PCR product on an agarose gel. No product band should be observed for any other primer set. This was the case for the WT genomic DNA template. If any sort of incorporation has occurred in the genome, one should be able to readily amplify out the TH marker with primer set 2 and resolve an ˜1000 bp PCR product. This is what was observed from both mutant DNA templates, and there was no product for WT template, as expected. If integration occurred through the first region of homology, one should readily amplify a ˜1700 bp product with primer set 3. This PCR product includes the 5′-flanking region of the chromosome, the first region of homology and the entire TH marker. If integration occurred through the first region of homology one could also theoretically amplify a >5000 bp region with primer set 4. This PCR product consists of the 3′-flanking region of the chromosome, the entire 3′-coding region of the gene up to the point where the first region of homology incorporated, the vector backbone, the second region of homology and the TH marker. If integration occurred through the second region of homology, a ˜1700 bp product with primer set 4 should be observed. This PCR product consists of the 3′-flanking region of the chromosome, the second region of homology and the TH marker. A >5000 bp region is also amplified with primer set 3. This PCR product would consist of the 5′-flanking region of the chromosome, the coding region of the gene to where the second region of homology ends, the vector backbone, the first region of homology and the TH marker. The >5000 bp products are not likely to amplify because the small PCR product will out compete the large PCR production for dNTPs. Thus, if integration has occurred, the expected results are no product band primer set 1, an intense product band for primer set 2 and a single intense product band for either primer set 3 or 4 but not both. For both mutant DNA templates the results indicate single integration through the first region of homology, refer to FIG. 11.

In order to definitively confirm, PCR amplification of regions about the chromosome that extend into the plasmid integrated DNA was performed and the results were sequenced. Sequencing primer sets are provided in Table 5. Sequencing results conclusively proved a single integration through the first region of homology.

Phenotypic Results from sigE Single Crossover Disruption

Morphology was observed via phase contrast microscopy to the known sigE deletion mutant phenotype of the best-studied relative, B. subtilis. There exist readily identified homologs to all the important sporulation associated sigma factors from B. subtilis in C. acetobutylcum (Paredes et al., Nat Rev Microbiol, 2005. 3(12): p. 969-78). In the case of a sigE disruption in B. subtilis, cells are arrested at early the forespore stage of sporulation (Peters et al., J Bacteriol, 1992. 174(14): p. 4629-37). Thus an asporogenous phenotype was confirmed via phase contrast microscopy and flow cytometry.

### Example 4

### Site-Specific Recombination for Double or Single Crossover Gene Knock-In

The previous examples are both examples of gene knock-ins in Clostridia. Although not commonly thought of as gene knock-ins, the integration of a foreign antibiotic selection marker is a gene knock-in.

Embodiments of the present invention reliably generates single and/or double crossovers precisely through the designed regions of homology, and all subsequent integrated DNA has been incorporated into the chromosome without any sequence deletions or rearrangements. None of the previously reported gene disruptions in C. acetobutylicum accomplished via homologous recombination ever reported the actual sequence data for the region of integration. Moreover, subsequent analysis of the previously reported spo0A disruption mutant indicated that integration did not take place via the two designated regions of homology. Additionally, the second crossover event appears to be more of a stochastic event, thus integration is likely not going to routinely generate perfect gene knock-ins.

The recently reported ClosTron system is limited in the length of DNA it can integrate into the site of gene disruption. Sigma-Aldrich reports the length limitation to be less than 2 Kb, and additionally admits this to be a significant limitation of the TargeTron™ system. The ClosTron system is further limited because the majority of the 2 Kb is already consumed by the selectable EM marker. Through the sigE single crossover gene disruption the above example demonstrates the ability to integrate more than 5 Kb of foreign DNA into the chromosome, which is plenty for integrating large synthetic gene operons or majority of DNA sequences of interest.

### Example 5

### Inducing Genetic Heterogeneity or Generating Genetic Diversity Through Resolvase Induced Chromosomal Recombination and/or Mutation Events

Construction of pRecU

The construction of pRecU was described previously in Example 1. The pRecU was transformed in C. acetobutylicum via electroporation described earlier and maintained with EM selection.

### Inducing Genetic Heterogeneity, Mutant Screening and Mutant Enrichment

The pRecU strain was grown in the presence of a sub-lethal concentration of butanol (˜1.0%) until mid-stationary phase. Upon reaching mid-stationary phase (˜24 hours of growth), the culture was used to inoculate a fresh flask containing no butanol. This process of alternating between growth in a flask containing butanol (which increased in concentration up to 1.9% butanol with each successive transfer into butanol-containing media) and then in a flask containing no butanol was continued until the culture ceased growth due to the high butanol concentration (previous attempts at C. acetobutylicum enrichment have proved successful only to an ultimate concentration of 1.6% butanol (Borden and Papoutsakis, Appl Environ Microbiol, 2007. 73(9): p. 3061-8). The purpose of alternating between selective and non-selective growth conditions is to increase the diversity of phenotypes selected. Selection in media containing butanol enriches for butanol-tolerant and butanol-overproducing mutations. Alternatively, selection in butanol-free media enriches for faster growth and asporogenous mutations.

After transferring into media containing 1.7% butanol, plates were streaked in order to select individual colonies and confer the desired phenotypes (the enrichment process was continued into media containing 1.9% butanol, as described above). To investigate butanol tolerance, butanol overproduction, and/or loss of sporulation, over 30 individual colonies from the culture containing 1.7% butanol were grown in test tubes, as well as 5 colonies of C. acetobutylicum (pRecU) that had not undergone any enrichment. These 35 flasks and test tubes were sampled and analyzed by HPLC to quantify butanol production. Flasks that showed butanol over-production were also sampled for phase contrast microscopy analysis; to identify if spores were present.

### HPLC Solvent Analysis for Mutants Versus Plasmid Control

Twenty four of the colonies selected did not produce appreciable amounts of butanol (e.g., final butanol concentrations <50 mM). However, six of the colonies selected showed moderate to large increases in butanol production above what the original strain was capable of producing. For instance, the 6^{th} colony selected from the 1.7% culture (i.e., sample 1.7% #06 in the Table below) produced 186.7 mM butanol, while the RecU strain produced 151.5 mM, on average, or an increase of 23%. Overall, the six over-producing strains demonstrated an average of an 11% increase in butanol titer over the original, non-mutated/non-enriched strain. Refer to Table 3 for results.

### Phase Contrast Microscopy Analysis of Mutant Versus Plasmid Control

Several butanol over-producing strains, generated by the enrichment process and described above, were compared by microscopy to C. acetobutylicum (pRecU) that had not undergone enrichment.

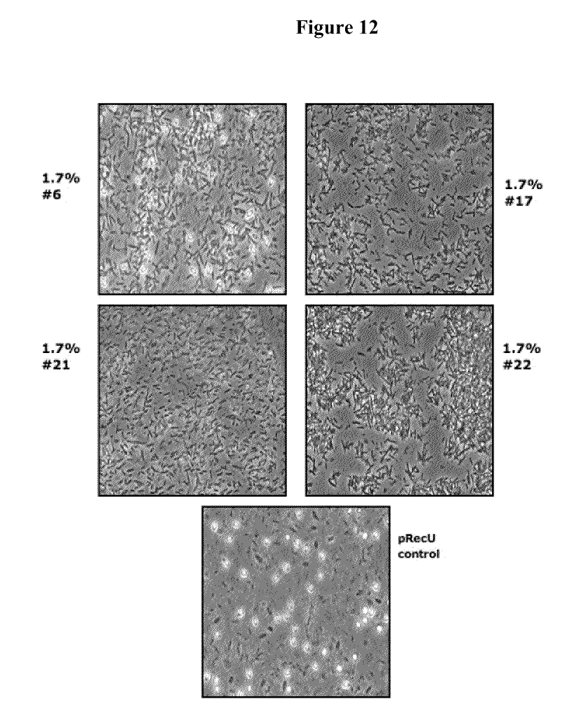

The strain of C. acetobutylicum (pRecU) that had not undergone enrichment was able to produce both spores and solvents. This is expected because no opportunity has been provided for either the generation of random genetic mutations, or the selection and enrichment for mutations conferring the desired phenotype. Five of the six enriched, mutant strains that showed enhanced solventogenesis and increased butanol tolerance, also demonstrated an asporogenous phenotype. Only the 1.7% #6 sample showed both solventogenesis and sporulation. Microscopy results are given in FIG. 12.

### Discussion of Results

From these images and the HPLC data presented above, it is apparent that multiple genetic mutations have occurred to bring about the multiple observed phenotypes. The first phenotype is the result of a type 1 genetic mutation that allows for increased solvent tolerance and production. This is evident because of the increased production potential and butanol tolerance compared to that of the unenriched C. acetobutylicum (pRecU) strain.

A second independent mutation (type 2), occurred in strains 17% #17, 21, 22, 23, and 26, resulting in an asporogenous phenotype. It is contemplated that two types of mutations have occurred because of the existence of the 1.7% #6 strain. This strain in fact produces the highest butanol titers (due to a type 1 mutation), but continues to generate spores (due to the lack of a type 2 mutation).

TABLE 1
Protein homology search results between model organism B. subtilis
ATCC23857 and C. acetobutylicum ATCC824. Results clearly show
that C. acetobutylicum homologous recombination machinery is
resolvase deficient.

| | | | B. subtilis |
|---|---|---|---|
| | | C. acetobutylicum | (ATCC23857) |
| Role | Name | (ATCC824) gene | gene |
| | addA | CAC2262 | BSU10630 |
| | addB | CAC2263 | BSU10620 |
| Pre-synaptic proteins | recD | CAC2854 | BSU27480 |
| (strand invasion) | recF | CAC0004 | BSU00040 |
| | recO | CAC1309 | BSU25280 |
| | recR | CAC0127 | BSU00210 |
| | recJ | CAC1198 | BSU32090 |
| | recN | CAC2073 | BSU24240 |
| | recQ | CAC2687 | BSU23020 |
| Strand exchange proteins | recA | CAC1815 | BSU16940 |
| | recG | CAC1736 | BSU15870 |
| | ruvA | CAC2285 | BSU27740 |
| | ruvB | CAC2284 | BSU27730 |
| Resolvase | RecU | ** | BSU22310 |
| Anti-recombination proteins | sbcC | CAC2736 | BSU10650 |
| | sbcD | CAC2737 | BSU10640 |
| | mutS | CAC1837 | BSU17040 |
| | mutS1 | CAC2340 | BSU28580 |
| | mutL | CAC1836 | BSU17050 |

** There is no annotated gene

TABLE 2
Additional protein homology search results.

| | Role | | | | | |
|---|---|---|---|---|---|---|
| | | | | | | Strand |
| | | | | | | exchange |
| | Initiation proteins | | Pre-synaptic proteins (strand invasion) | | | proteins |
| Name | addA | addB | recF | recO | recR | recA |
| B. subtilis | BSU10630 | BSU10620 | BSU00040 | BSU25280 | BSU00210 | BSU16940 |
| (ATCC23857) | | | | | | |
| gene | | | | | | |
| C. acetobutylicum | CAC2262 | CAC2263 | CAC0004 | CAC1309 | CAC0127 | CAC1815 |
| (ATCC824) gene | | | | | | |
| C. difficile 630 | CD0328 | CD1040 | CD0004 | CD2435 | CD0018 | CD1328 |
| C. perfringens | CPE0021 | CPE0020 | CPE0004 | CPE2014 | CPE0047 | CPE1673 |
| strain 13 | | | | | | |
| C. tetani E88 | CTC00714 | CTC00686 | CTC00092 | CTC02017 | CTC00073 | CTC01289 |
| C. novyi NT | NT01CX_1236 | NT01CX_1235 | NT01CX_0864 | NT01CX_0042 | NT01CX_0823 | NT01CX_2123 |
| C. beijerincki | CbeiDRAFT_2818 | CbeiDRAFT_2819 | CbeiDRAFT_0674 | CbeiDRAFT_4216 | CbeiDRAFT_1313 | CbeiDRAFT_0310 |
| NCIMB 8052 | | | | | | |
| C. thermocellum | Cthe_2039 | Cthe_2040 | Cthe_2374 | Cthe_1066 | Cthe_2142 | Cthe_1050 |
| ATCC27405 | | | | | | |
| C. cellulolyticum | CcelDRAFT_1473 | CcelDRAFT_1471 | CcelDRAFT_2615 | CcelDRAFT_3134 | CcelDRAFT_2404 | CcelDRAFT_1632 |
| H10 | | | | | | |
| C. phytofermentas | CphyDRAFT_1356 | CphyDRAFT_1357 | CphyDRAFT_2521 | CphyDRAFT_1607 | CphyDRAFT_2219 | ** |
| ISDg | | | | | | |
| | | Role | | | | |
| | | Strand exchange | | | Anti-recombination | |
| | | proteins | | Resolvase | proteins | |
| | Name | ruvA | ruvB | RecU | mutS | mutS1 |
| | B. subtilis | BSU27740 | BSU27730 | BSU22310 | BSU17040 | BSU28580 |
| | (ATCC23857) | | | | | |
| | gene | | | | | |
| | C. acetobutylicum | CAC2285 | CAC2284 | ** | CAC1837 | CAC2340 |
| | (ATCC824) gene | | | | | |
| | C. difficile 630 | CD2806 | CD2805 | ** | CD1977 | CD0709 |
| | C. perfringens | CPE1948 | CPE1947 | ** | CPE1155 | CPE1881 |
| | strain 13 | | | | | |
| | C. tetani E88 | CTC02212 | CTC02211 | ** | CTC01302 | CTC02274 |
| | C. novyi NT | NT01CX_1832 | NT01CX_1833 | ** | NT01CX_2105 | NT01CX_1773 |
| | C. beijerincki | CbeiDRAFT_4890 | CbeiDRAFT_4891 | ** | CbeiDRAFT_2634 | CbeiDRAFT_4312 |
| | NCIMB 8052 | | | | | |
| | C. thermocellum | Cthe_0181 | Cthe_0182 | ** | Cthe_0777 | Cthe_1014 |
| | ATCC27405 | | | | | |
| | C. cellulolyticum | ** | ** | ** | CcelDRAFT_1548 | CcelDRAFT_0051 |
| | H10 | | | | | |
| | C. phytofermentas | CphyDRAFT_0104 | CphyDRAFT_0105 | CphyDRAFT_1256 | CphyDRAFT_0638 | CphyDRAFT_2693 |
| | ISDg | | | | | |

Protein homology search results of essential homologous recombination machinery from B. subtilis compared to additional solventogenic, pathogenic and industrial relevant strains of Clostridia. Results indicate that the majority of Clostridia are resolvase deficient.
** indicates that there is no orthology to the respective B. subtilis protein.

TABLE 3
HPLC results from enriched and selected pRecU mutants. Six of the
isolated mutants were butanol over-producing strains compared to the
non-enriched pRecU control strain. The increase in production compared
to control varies, thus the responsible mutations are likely diverse.

| Sample | Butanol | % Increase over RecU Avg |
|---|---|---|
| RecU #1 | 144.6 | |
| RecU #2 | 148.8 | |
| RecU #3 | 162.4 | |
| RecU #4 | 140.0 | |
| RecU #5 | 161.6 | |
| | 151.5 | |
| 1.7% #06 | 186.7 | 23% |
| 1.7% #17 | 160.7 | 6% |
| 1.7% #21 | 180.1 | 19% |
| 1.7% #22 | 161.9 | 7% |
| 1.7% #23 | 162.1 | 7% |
| 1.7% #26 | 157.8 | 4% |
| | 168.2 | 11% |

TABLE 4
Table of strains and plasmids employed in this study.

| Strain or Plasmid Name | Relevant Characteristics | Source |
|---|---|---|
| Strain | | |
| E. coli One Shot Chemically | Invitrogen competent cells | Invitrogen |
| Competent TOP10 | | |
| E. coli ER2275 | recA IacZ mcrBC | NEB |
| ATCC824 | type strain | ATCC |
| SKO1 | ATCC824 spo0A::MLS^{r} | Harris et al. |
| SKO mutants | ATCC824 spo0A::MLS^{r} | this study |
| BTSIGE | ATCC824 sigE::Th^{r} | this study |
| Plasmids | | |
| pSOS95 | Amp^{r} MLS^{r}; repL ori; ace2 operon under thL promoter | Tummala et al. 1999 |
| pSOS95del | Amp^{r} MLS^{r}; repL ori; thL promoter | Tummala et al. 2003 |
| pSOS94 | Amp^{r} MLS^{r}; repL ori; ace2 operon under ptB promoter | Tummala et al. 1999 |
| pSOS94del | Amp^{r} MLS^{r}; repL ori; ptB promoter | Tummala et al. 1999 |
| pETSPO | Th^{r}; repL ori; spo0A::MLS^{r} | Harris et al. |
| pRecU | Amp^{r} MLS^{r}; repL ori; recU under thL promoter | this study |
| pAN2 | Amp^{r}; carries the φ3TI gene | Tomas, C. (unpublished) |
| pCR8-GW-TOPOTA | Sp^{r}; topoisomerized; ori | Invitrogen |
| pCR8-SigE | pCR8-GW-TOPOTA withsigE fragmentcloned | this study |
| pCR8-GW-SigE/CM/ptB | pCR8-GW-TOPOTA withsigE::modified Th^{r} | this study |
| pKORSPO0A | Amp^{r} MLS^{r}; repL ori; recU under thL promoter; spo0A::Th^{r} | this study |
| pKORSIGE | Amp^{r} MLS^{r}; repL ori; recU under thL promoter; sigE::Th^{r} | this study |
| pLHKO | Th^{r}; repL ori | Harris et al. |
| p95CM | Amp^{r} MLS^{r}; repL ori; Cm/Th^{r} under thL promoter | this study |
| p94CM | Amp^{r} MLS^{r}; repL ori; Cm/Th^{r} under ptB promoter | this study |

ace2 operon, synthetic operon which contains the three acetone formation genes (adc, ctfA, and ctfB) transcribed from the adc promoter from ATCC824 (AE001437);
Amp^{r}, ampicilin resistance gene;
DEST cassette, Invitrogen Destination cassette for Gateway ™ cloning system;
MLS^{r}, macrolide-lincosamide-streptogramin resistance gene;
repL, pIM13 gram-positive origin of replication;
ori, ColE1 origin of replication;
recU, resolvase ORF and Shine-Delgarno sequence (BSU22310) from B. subtilis ATCC23857 (GenBank# AL009126;
Refseq NC_000964);
Sp^{r}, spectinomycin resistance gene;
Th^{r}, thiamphenicol and chloramphenicol resistance gene;
φ3TI, Bacillus subtilis phage φ 3TI methyltransferase gene
NEB, New England Biolabs, Beverly, MA.
ATCC, American Type Culture Collection, Manassas, VA.

TABLE 5
Table of Primer Sequences employed in this study.

| | | | |
|---|---|---|---|
| Sequence Name | Sequence (5′-3′) | Description | Sequence ID NO |

| | | | |
|---|---|---|---|
| recU-F | CGGGATCCCGTCATGATTAGTTTAATAAGGA | FP to amplify the recU gene (BSU22310) | 2 |
| | GGATGA | from B. subtilis ATCC23857 genomic DNA | |
| | | (GenBank# AL009126; Refseq NC_000964) and | |
| | | a BamHI endonuclease recognition site | |
| recU-R | CGGCGCCGCTTCACGGCTGTTAAATTGATCT | RP to amplify the recU gene (BSU22310) | 3 |
| | | from B. subtilis ATCC23857 genomic DNA | |
| | | (GenBank# AL009126; Refseq NC_000964) | |
| | | and a KasI endonuclease recognition site | |
| recU-cass-F | GGAATGGCGTGTGTGTTAGCCAAA | FP to amplify recU out of pSOS94del or | 4 |
| | | pSOS95del | |
| recU-cass-R | TCACACAGGAAACAGCTATGACCA | RP to amplify recU out ot pSOS94del or | |
| | | pSOS95del | |
| SigE-F | ATAGGTGGAAATGATGCGCTTCCG | FP to amplify a portion of CAC1695 from | 5 |
| | | C. acetobutylicum ATCC824 genomic DNA | |
| | | (GenBank# AE001437; Refseq NC_003030) | |
| SigE-R | CCCAGCATATCTGCAACTTCCT | RP to amplify a portion of CAC1695 from | 6 |
| | | C. acetobutylicum ATCC824 genomic DNA | |
| | | (GenBank# AE001437; Refseq NC_003030) | |
| CM-F | TCGCTTCACGAATGCGGTTATCTC | FP to amplify 1567 bp | 7 |
| | | Chloramphenicol/Thiamphenicol | |
| | | antibiotic gene | |
| CM-R | CCAACTTAATCGCCTTCGAGCACA | RP to amplify 1567 bp | 8 |
| | | Chloramphenicol/Thiamphenicol | |
| | | antibiotic gene | |
| mod-CM/SDG-F | CCGGATCCACTTGAATTTAAAAGGAGGGAA | FP to amplify 687 bp novel | 9 |
| | CTTAGATGGTATTTGAAAAAATTGAT | Chloramphenicol/Thiamphenicol | |
| | | antibiotic gene | |
| mod-CM/SDG-R | CGGCGCCAGTTACAGACAAACCTGAAGT | RP to amplify bp novel | 10 |
| | | Chloramphenicol/Thiamphenicol | |
| | | antibiotic gene | |
| SigE-KO-conf-F | CGGCGCCAGTTACAGACAAACCTGAAGT | FP to confirm SigE gene disruption | 11 |
| SigE-KO-conf-R | CTGGCAGTTGTGTTTCCATTCCTC | RP to confirm SigE gene disruption | 12 |
| Spo0A-KO-conf-F | GTCTCAAATCATTATATACAGCCC | FP to confirm Spo0A gene disruption | 13 |
| Spo0A-KO-conf-R | TGGGAAATTTAATGTTGTGGAAGA | RP to confirm Spo0A gene disruption | 14 |
| SigE-Seq-PS1-F | TGGCGCCACTTAATGATTTGCCAG | SigE integration sequencing PS1 F | 15 |
| SigE-Seq-PS1-R | TATCTGACGTCAATGCCGAGCGAA | SigE integration sequencing PS1 R | 16 |
| SigE-Seq-PS2-F | TGGAAAGGCAGGTAACCTTGAAGC | SigE integration sequencing PS2 F | 17 |
| SigE-Seq-PS2-R | AGCAGCTTGTTTCCATCCCAGTCT | SigE integration sequencing PS2 R | 18 |
| SigE-Seq-PS3-F | TAAATGCTACCCTTCGGCTCGCTT | SigE integration sequencing PS3 F | 19 |
| SigE-Seq-PS3-R | ATCTTCGAGGGTCATTCCGCGATT | SigE integration sequencing PS3 R | 20 |
| SigE-Seq-PS4-F | GCCGAAACATTCGGTTTCATCCCA | SigE integration sequencing PS4 F | 21 |
| SigE-Seq-PS4-R | TGGTTTGTTTGCCGGATCAAGAGC | SigE integration sequencing PS4 R | 22 |
| SigE-Seq-PS5-F | GCTCTTGATCCGGCAAACAAACCA | SigE integration sequencing PS5 F | 23 |
| SigE-Seq-PS5-R | CTGGCAGTTGTGTTTCCATTCCTC | SigE integration sequencing PS5 R | 24 |

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. A method for incorporating genetic material into a bacterial genome, wherein said bacterial genome lacks a functional resolvase gene, comprising: contacting a bacterial cell comprising a bacterial genome with at least one plasmid comprising a gene encoding a resolvase protein and a nucleic acid of interest under conditions such that said nucleic acid of interest integrates into said bacterial genome.

2. The method of claim 1, wherein said bacterial cell is Clostridia cell.

3. The method of claim 1, wherein said gene encoding a resolvase protein and said nucleic acid of interest are on the same plasmid.

4. The method of claim 1, wherein said gene encoding a resolvase protein and said nucleic acid of interest are on two distinct plasmids.

5. The method of claim 1, wherein said nucleic acid of interest integrates into said bacterial genome via homologous recombination.

6. The method of claim 5, wherein said homologous recombination is site specific recombination.

7. The method of claim 1, wherein said integration of said nucleic acid of interest into said bacterial genome results in disruption of function of one or more genes in said bacterial genome.

8. The method of claim 1 wherein said resolvase polypeptide is encoded by the recU gene from Bacillus subtilis.

9. The method of claim 8, wherein said recU gene has the nucleic acid sequence described by SEQ ID NO:25.

10. The method of claim 1, wherein said resolvase gene is under the control of a Clostridia promoter.

11. The method of claim 10, wherein said Clostridia promoter is selected from the group consisting of a Clostridium thiolase (thL) and a phosphotransbutyrylase (ptB) promoters.

12. The method of claim 1, wherein said nucleic acid of interest encodes a selectable marker.

13. The method of claim 13, wherein said selectable marker is an antibiotic resistance gene.

14. A method, comprising: contacting a bacterial cell comprising a bacterial genome lacking a native resolvase gene with a nucleic acid encoding an exogenous resolvase gene under conditions such that said exogenous resolvase gene is stability incorporated into said bacterial cell.

15. The method of claim 14, further comprising the step of contacting said bacterial genome with a sub-lethal concentration of a reagent that induces mutation.

16. The method of claim 14, further comprising the step of selecting for bacterial cells that grow in the presence of said reagent.

17. The method of claim 14, wherein said exogenous resolvase gene is stability incorporated into said bacterial cell via a plasmid or incorporation into the genome of said bacterial cell.

18. A bacterial cell comprising an exogenous nucleic acid encoding a resolvase protein.

19. The bacterial cell of claim 18, wherein said bacterial cell lacks a native resolvase gene.

20. The bacterial cell of claim 18, wherein said resolvase gene has the nucleic acid sequence of SEQ ID NO:25 or encodes a protein having an amino acid sequence selected from the group consisting of: SEQ ID NOs: 26-33.

21. The bacterial cell of claim 18, wherein said resolvase gene is present on a plasmid.

22. The bacterial cell of claim 18, wherein said resolvase gene is integrated into the genome of said bacterial cell.


SEQUENCE LISTING (34 sequences)

SEQ ID NO: 1 - DNA, 1844 bp, Artificial Sequence
  Synthetic
taagtcttagcttgtcagcaatcatagcaataaattcactattagtaggtttacctttgccattgttgatagtatatccaaataacttatttatagtttcaacctgtcccggaattcggatccacgtgaccatgcaacttcaatagcatgtcttattgctctttctactctgctagctgtagtattgtatttttttgcaatagaaggatataattccttagtaactgctgataaaagctccatattattaactaccattgttatagcttcccttaaatacatataaccttttatatgtgctggaacacctatttgatgtattattgatgtaatttcgctttctaaatcaaccgctttattacctatattatcagagtaaacatcatttgcaattgtacctgcaaagcttgtgaatgcgcaaaagacataatcgattcacaaaaaataggtacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttctgtttatctttgtgtgatattcttgatttttttctatttaatctgataagtgagctattcactttaggtttaggatgaaaatattctcttggaaccatacttaatatagaaatatcaacttctgccattaaaaataatgccaatgagcgttttgtatttaataatcttttagcaaacccgtattccacgattaaataaatctcatcagctatactatcaaaaacaattttgcgtattatatccgtacttatgttataaggtatattaccaaatattttataggattggtttttaggaaatttaaactgcaatatatccttgtttaaaacttggaaattatcgtgatcaacaagtttattttctgtagttttgcataatttatggtctatttcaatggcagttacgaaattacacctctgtactaattcaagggtaaaatgcccttttcctgagccgatttcaaagatattatcatgttcatttaatcttatatttgtcattattttatctatattatgttttgaagtaataaagttttgactgtgttttatatttttctcgttcattataaccctctttattttttcctccttataaaattagtataattatagcacgagctctgataaatatgaacatgatgagtgatcgttaaatttatactgcaatctgatgcgattattgaataaaagatatgagagatttatctagtttctttttttacaagaaaaaagaaagttcttaaaggttttatacttttggtcgtagagcacaagcttgcctctttctcttcaacttgatatgaccttttctgctctgaatttgatatagtattattaaacatttctcttattctattagtgaatacatccatatcaaaaggtttaactacatagtagtccgcccctaatgttatagctctttgtgttattttatcctgtccaacagcggataaaactattattcttggaaggttttctgcatctttattgttaagtttttctaatactcctagtccatctaatcttggcattattatatcgagaacaacaaggtcaggcggatccatttatatcgagaacaacaaggtcaggctttttattctctataagctttaaagcttccactccatcctttgctattccaacaactatcatatcgctttggttaagcaagtaatc

SEQ ID NO: 2 - DNA, 37 bp, Artificial Sequence
  Synthetic
cgggatcccgtcatgattagtttaataaggaggatga

SEQ ID NO: 3 - DNA, 31 bp, Artificial Sequence
  Synthetic
cggcgccgcttcacggctgttaaattgatct

SEQ ID NO: 4 - DNA, 24 bp, Artificial Sequence
  Synthetic
ggaatggcgtgtgtgttagccaaa

SEQ ID NO: 5 - DNA, 24 bp, Artificial Sequence
  Synthetic
ataggtggaaatgatgcgcttccg

SEQ ID NO: 6 - DNA, 22 bp, Artificial Sequence
  Synthetic
cccagcatatctgcaacttcct

SEQ ID NO: 7 - DNA, 24 bp, Artificial Sequence
  Synthetic
tcgcttcacgaatgcggttatctc

SEQ ID NO: 8 - DNA, 24 bp, Artificial Sequence
  Synthetic
ccaacttaatcgccttcgagcaca

SEQ ID NO: 9 - DNA, 56 bp, Artificial Sequence
  Synthetic
ccggatccacttgaatttaaaaggagggaacttagatggtatttgaaaaaattgat

SEQ ID NO: 10 - DNA, 28 bp, Artificial Sequence
  Synthetic
cggcgccagttacagacaaacctgaagt

SEQ ID NO: 11 - DNA, 24 bp, Artificial Sequence
  Synthetic
tggaaaggcaggtaaccttgaagc

SEQ ID NO: 12 - DNA, 24 bp, Artificial Sequence
  Synthetic
ctggcagttgtgtttccattcctc

SEQ ID NO: 13 - DNA, 24 bp, Artificial Sequence
  Synthetic
gtctcaaatcattatatacagccc

SEQ ID NO: 14 - DNA, 24 bp, Artificial Sequence
  Synthetic
tgggaaatttaatgttgtggaaga

SEQ ID NO: 15 - DNA, 24 bp, Artificial Sequence
  Synthetic
tggcgccacttaatgatttgccag

SEQ ID NO: 16 - DNA, 24 bp, Artificial Sequence
  Synthetic
tatctgacgtcaatgccgagcgaa

SEQ ID NO: 17 - DNA, 24 bp, Artificial Sequence
  Synthetic
tggaaaggcaggtaaccttgaagc

SEQ ID NO: 18 - DNA, 24 bp, Artificial Sequence
  Synthetic
agcagcttgtttccatcccagtct

SEQ ID NO: 19 - DNA, 24 bp, Artificial Sequence
  Synthetic
taaatgctacccttcggctcgctt

SEQ ID NO: 20 - DNA, 24 bp, Artificial Sequence
  Synthetic
atcttcgagggtcattccgcgatt

SEQ ID NO: 21 - DNA, 24 bp, Artificial Sequence
  Synthetic
gccgaaacattcggtttcatccca

SEQ ID NO: 22 - DNA, 24 bp, Artificial Sequence
  Synthetic
tggtttgtttgccggatcaagagc

SEQ ID NO: 23 - DNA, 24 bp, Artificial Sequence
  Synthetic
gctcttgatccggcaaacaaacca

SEQ ID NO: 24 - DNA, 24 bp, Artificial Sequence
  Synthetic
ctggcagttgtgtttccattcctc

SEQ ID NO: 25 - DNA, 621 bp, Bacillus subtilis
gtgattcggtatcctaatggaaaaacatttcagccgaaacattcggtttcatcccaaaacagtcagaaaagggccccgtcttacagtaatcgcggaatgaccctcgaagatgacttaaacgaaacgaataagtattatctgacaaaccaaattgccgttatacacaaaaagccgacacctgttcaaattgtaaatgtccattatccaaaaagaagtgccgcagtgattaaagaagcttactttaaacaatcatcgacaacagactacaatgggatttacaaagggcggtatattgattttgaggcgaaagaaacgaaaaacaagacctctttccccctgcagaattttcatgaccatcaaatcgagcatatgaagcaggtaaaggctcaagacggtatttgttttgttattatatccgctttcgaccaggtttattttttggaagccgataagctgttttatttctgggacagaaaagaaaaaaacggcagaaaatcaattcgaaaagatgagttggaagaaacagcttatccgatttctcttggatacgcacccagaattgattatattagtattattgaacagctttatttttcgccatcatctggtgcgaaaggttga

SEQ ID NO: 26 - protein, 143 aa, Artificial Sequence
  Synthetic
MNAKKRKGSAVERNIVSRLRDKGFAVVRAPASGSKRKDPIPDIIALKNGVIILIEMKSRKDIEGKIYVRREQAEGIIEFARKSGGSLFLGVKKPGVLKFIPFEKLRRTETGNYVADSEIEGLDLEDLVRLVEAKISRTLDNFL

SEQ ID NO: 27 - protein, 149 aa, Artificial Sequence
  Synthetic
MAGYGAKGIRKVGAFRSGLEDKVSKQLESKGIKFEYEEWKVPYVIPASNHTYTPDFLLPNGIFVETKGLWESDDRKKHLLIREQHPELDIRIVFSSSRTKLYKGSPTSYGEFCEKHGIKFADKLIPAEWIKEPKKEVPFDRLKRKGGKK

SEQ ID NO: 28 - protein, 173 aa, Artificial Sequence
  Synthetic
MAIILGIDPGSRVTGYGVIRQVGRQLSYLGSGCIRTKVDDLPSRLKLIYAGVTEIITQFQPDYFAIEQVFMAKNADSALKLGQARGVAIVAAVNQELPVFEYAARQVKQTVVGIGSAEKSQVQHMVRTLLKLPANPQADAADALAIAITHCHVSQNAMQMSESRLNLARGRLR

SEQ ID NO: 29 - protein, 353 aa, Artificial Sequence
  Synthetic
MSTAQKAKILQLIDSCCQNAKSTQLKSLSFVIGAVNGTTKEAKRTYIQEQCEFLEKLRQQKIREGRINILSMDAGVSNFAFSKMQLLNNDPLPKVLDWQKINLEEKFFQNLKKLSLNPAETSELVFNLTEYLFESMPIPDMFTIERQRTRTMSSRHILDPILKVNILEQILFSNLENKMKYTNKIPNTSKLRYMVCSSDPHRMTSYWCIPREETPTSSKKLKSNKHSKDSRIKLVKKILSTSILEGNSTSSTKLVEFIGVWNNRIRNALTKKKSFKLCDILEIQDNSGVRKDDDLADSFLHCLSWMEWLKNYESITELLNSKTLVKTQFGQVFEFCENKVQKLKFLQNTYNND

SEQ ID NO: 30 - protein, 176 aa, Artificial Sequence
  Synthetic
MSSPMSKKDYSSEIICAFDIGAKNPARTVLEVKDNSVRVLDISKLDWSSDWERRIAKDLSQYEYTTVLLERQPRRSPYVKFIYFIKGFLYHTSAAKVICVSPVMSGNSYRDRKKRSVEAFLDWMDTFGLRDSVPDRRKLDDVADSFNLAMRYVLDKWNTNYTPYNRCKSRNYIKKM

SEQ ID NO: 31 - protein, 9 aa, Artificial Sequence
  Synthetic
MNTYSITLP

SEQ ID NO: 32 - protein, 157 aa, Artificial Sequence
  Synthetic
MLLTGKLYKEEKQKFYDAQNGKCLICQRELNPDVQANHLDHDHELNGPKAGKVRGLLCNLCNAAEGQMKHKFNRSGLKGQGVDYLEWLENLLTYLKSDYTQNNIHPNFVGDKSKEFSRLGKEEMMAEMLQRGFEYNESDTKTQLIASFKKQLRKSLK

SEQ ID NO: 33 - protein, 206 aa, Artificial Sequence
  Synthetic
MIRYPNGKTFQPKHSVSSQNSQKRAPSYSNRGMTLEDDLNETNKYYLTNQIAVIHKKPTPVQIVNVHYPKRSAAVIKEAYFKQSSTTDYNGIYKGRYIDFEAKETKNKTSFPLQNFHDHQIEHMKQVKAQDGICFVIISAFDQVYFLEADKLFYFWDRKEKNGRKSIRKDELEETAYPISLGYAPRIDYISIIEQLYFSPSSGAKG

SEQ ID NO: 34 - DNA, 24 bp, Artificial Sequence
  Synthetic
tcacacaggaaacagctatgacca